(19) United States
(12) Plant Patent Application Publication
(10) Pub. No.: US 2005/0022275 P1
(43) Pub. Date: Jan. 27, 2005

(54) Clementine tree named 'Clemenpons'

(76) Inventor: Juan Pons Bolta, Castellon (ES)

Correspondence Address: WEBB ZIESENHEIM LOGSDON ORKIN & HANSON, P.C., 700 KOPPERS BUILDING, 436 SEVENTH AVENUE, PITTSBURGH, PA, 15219, US

(21) Appl. No.: 10/859,428

(22) Filed: Jun. 2, 2004

Related U.S. Application Data
(63) Continuation of application No. 10/047,911, filed on Jan. 15, 2002, now abandoned.

(51) Int. Cl. A01H 5/00; C11D 3/00; C11D 7/42; C12S 9/00

(52) U.S. Cl. PLT/202

(57) ABSTRACT

A new and distinct Citrus reticulata tree with large, early ripening fruit.

### CROSS-REFERENCE TO RELATED APPLICATIONS

This application is a continuation of co-pending U.S. Plant patent application Ser. No. 10/047,911 filed Jan. 15, 2002, which is hereby incorporated by reference in its entirety.

### BOTANICAL CLASSIFICATION

Citrus reticulata

### VARIETAL DENOMINATION

‘Clemenpons’

### BACKGROUND OF THE NEW PLANT

The present invention comprises a new and distinct cultivar of clementine tree botanically known as Citrus reticulata, and herein referred to by its cultivar name ‘Clemenpons’.

The new cultivar was discovered in 1964 as a seedling in Pego, Aligante, Spain as a spontaneous mutation of ‘de Nules’ clementine (unpatented). ‘Clemenpons’ differs from ‘de Nules’ in that it has an earlier fruit maturity and characteristic burr knots and galls on its trunk.

The new cultivar was first asexually reproduced in 1985 in Pego (Valencia), Spain by grafting budwood onto citrange rootstock (unpatented). Subsequent grafting of budwood onto citrange rootsock in Moncado (Valencia), Spain has shown the features of the new cultivar to be stable and reproduce true to type in successive propagations.

The following traits are determined to be basic characteristics of the new cultivar which in combination distinguish this clementine tree as new and distinct.

1. Earlier fruit maturity then ‘Clemenules’ (unpatented).

2. Fruit characteristics similar to ‘Clemenules’; superior fruit quality to ‘Arrufatina’ Clementine (unpatented).

3. Tree characteristics similar to ‘Clemenules’; less vigorous growth than ‘Fina’ (Commune) Clementine (unpatented).

4. Fruit internal maturity and rind colors up to 3 weeks earlier than ‘Clemenules’.

5. Larger fruit than ‘Fina’ (Commune) Clementine.

### DESCRIPTION OF THE DRAWINGS

The accompanying photographic drawings illustrate the new cultivar with the color being as true as possible with color illustrations of this type. The photographs were taken under natural light conditions.

FIG. 1 shows fruit of the new variety;

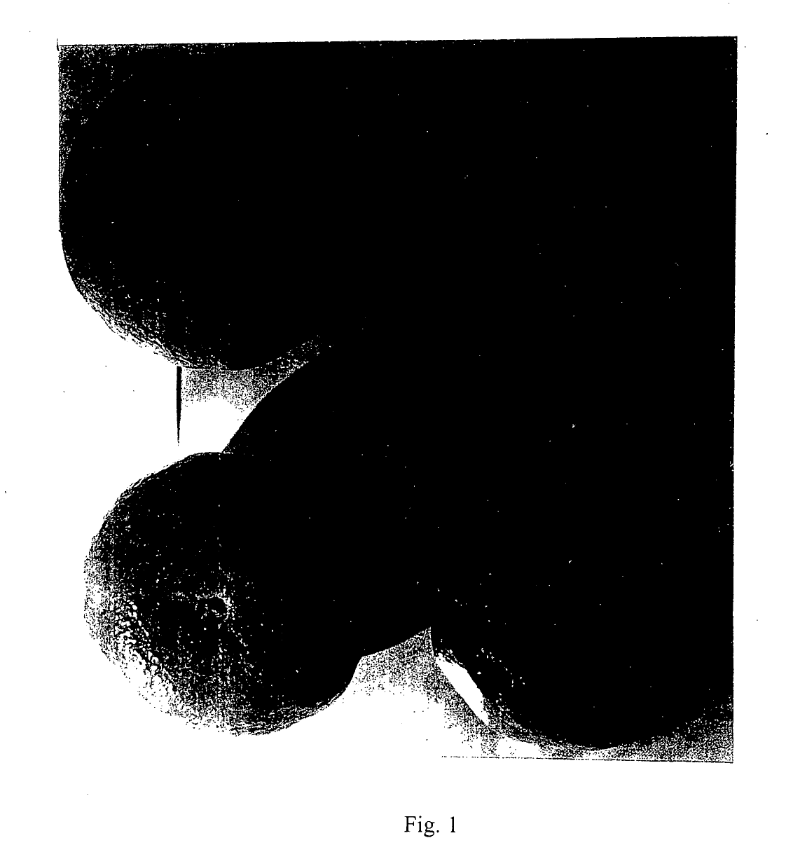

FIG. 2 shows the flesh of the new variety;

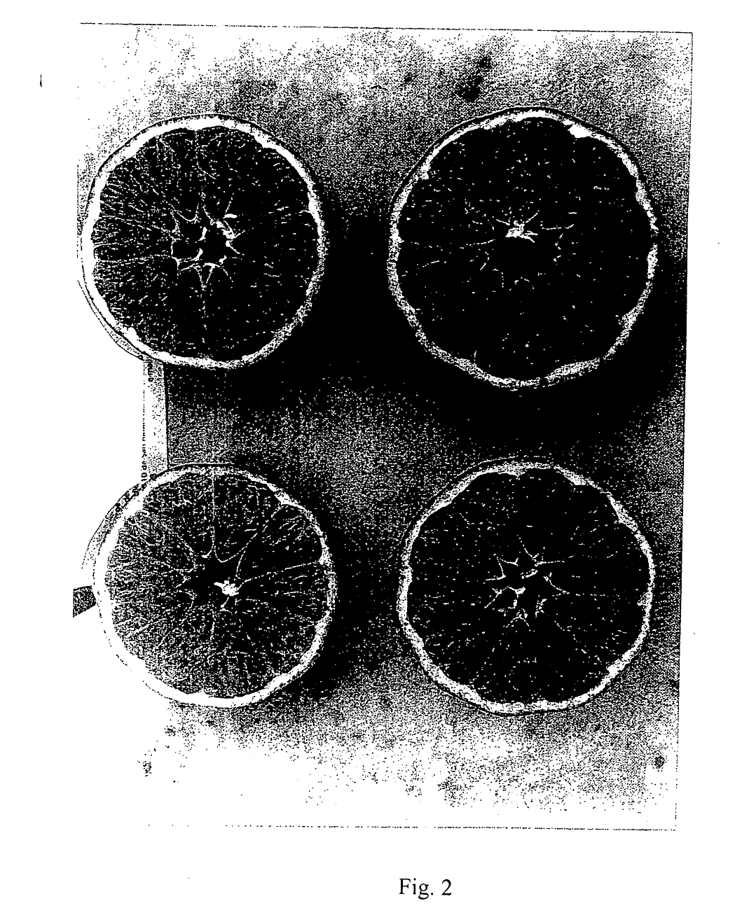

FIG. 3 shows color indices during ripening of the fruit, “1000 a/L.b (Parametros de Hunter)” is an index of color;

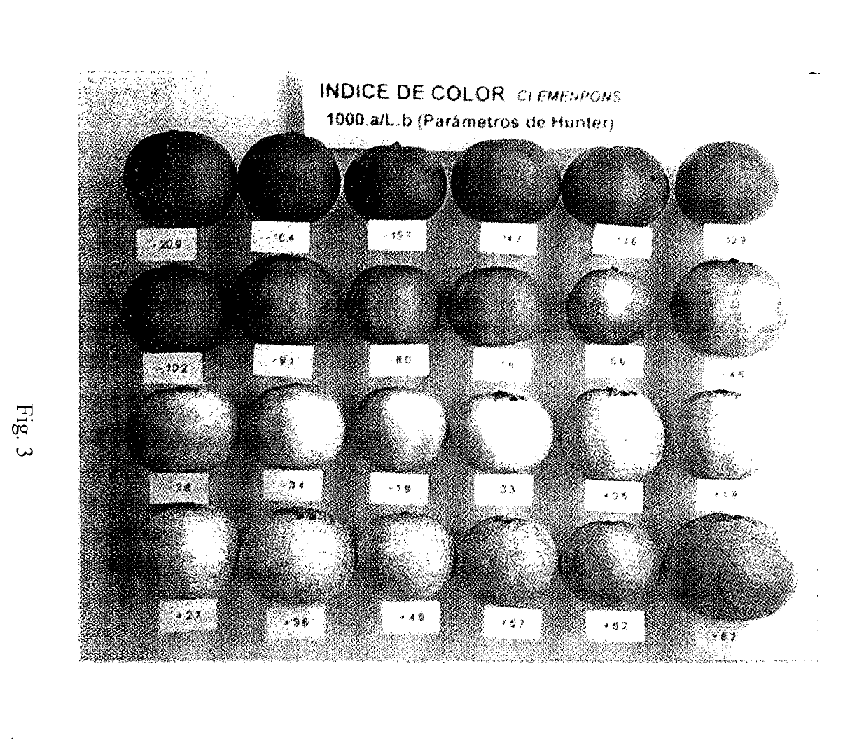

FIG. 4 shows the tree; and

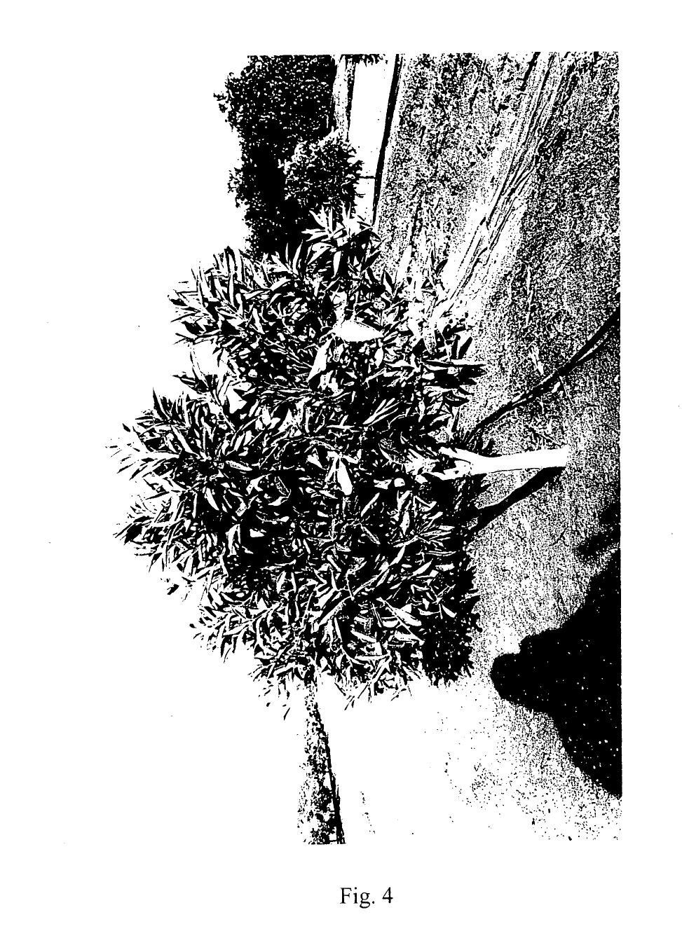

FIG. 5 shows the blossoms of the new variety.

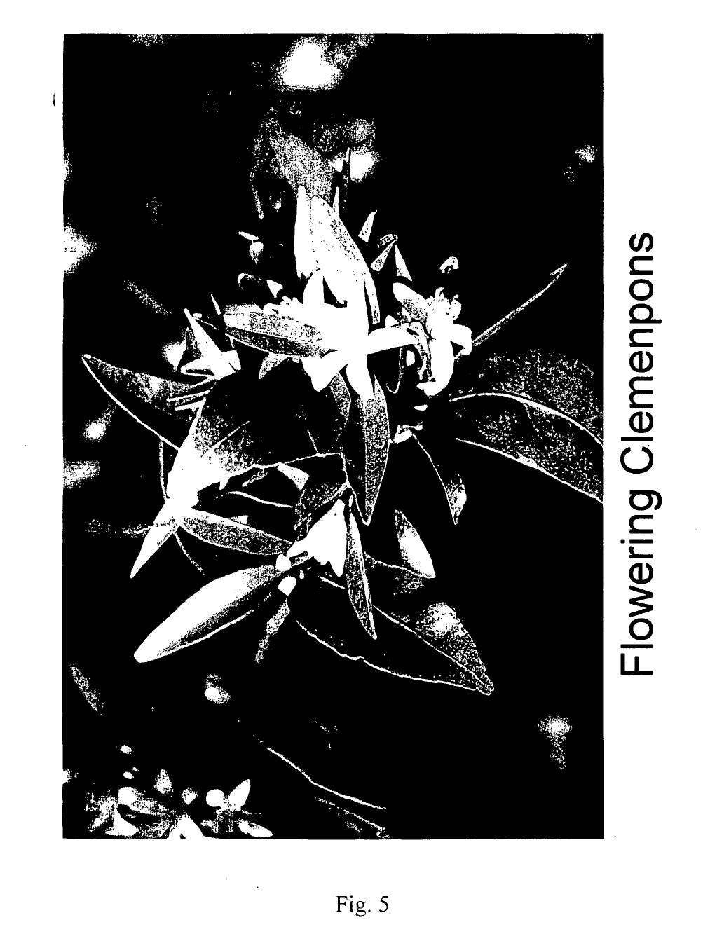

### DESCRIPTION OF THE PLANT

The following description sets forth the characteristics of the new cultivar. The data which defines these characteristics were collected from asexual reproductions by grafting of budwood onto Troyer citrange rootstock. The plant history was taken on a 5 year old tree outside in Moncada (Valencia), Spain. In the following description, color references are made to the R.H.S. Colour Chart of The Royal Horticultural Society of London.
Classification:
   Botanical.—Citrus reticulate.
   Commercial.—Clementine tree.
Tree:
   Growth rate.—Medium vigor; similar to Clemenules.
   Average amount of time to produce a fruit bearing tree.—From nursery to planting: 2 years. From planting to cropping: 3-4 years.
   Average amount of growth per season.—2 to 4 cycles of 6-12 inches.
   Overall shape.—Spreading.
   Height.—Up to 12 feet, if allowed.
   Spread.—Depending on spacing, up to 12 feet.
   Preferred soil type.—Varies with rootstock type; loamy.
   Bark color.—Young: Yellow-Green Group 146A. Old: Grey-Brown Group 199C.
Foliage:
   Shape.—Overall: Lanceolate. Base: Obtuse. Tip: Acute.
   Size.—Length: 61 mm. Width: 20 mm.
   Color.—Upper surface: Green Group 132A. Lower surface: Green Group 132B. Venation: Yellow-Green Group 144B.
   Marginal form.—Entire.
   Petiole.—Diameter: 1.2 mm. Length: 6 mm. Color: Yellow-Green Group 146A/B.
   Scent.—Pleasant.
   Surface texture.—Medium roughness.
Flowers:
   Blooming period.—April in Moncada (Valencia), Spain.
   Sepals.—Shape: Pyramidal. Number: 5. Length: 4.0 mm. Width: 2.5 mm. Apex: 0.5 mm. Margin: Smooth. Base width: 2.0 mm. Sepal color: Upper surface: Yellow-Green Group 146B. Lower surface: Yellow-Green Group 146B.
   Petal.—Shape: Elongated; tapered. Number: 5. Length: 12 mm. Width: 6 mm. Apex: Pointed. Margin: Smooth. Base Width: 5 mm. Petal color: Upper Surface: White Group 155A. Lower surface: White Group 155A.
   Pedicel length.—4 mm.
   Pedicel color.—Yellow-Green Group 146A/B.
   Overall size.—Bud: Length: About 3 mm. Diameter: About 2.5 mm. Opened flower: Length: About 5 mm. Diameter: About 2.5 mm.
   Bud color.—Yellow-Green Group 146A/B.
Fruit:
   Maturity date.—Mid-October in Moncada (Valencia), Spain.
   Weight.—70 to 120 g.
   Shape.—Overall: In dry climates, spherical; In coastal climates, oblate. Base: Slightly indented. Apex: Neck/nipple develops only in cold inland areas. Diameter: 55-65 mm. Height: 40-50 mm. Furrows: None.
Rind:
   Surface texture.—Smooth; slightly pebbly.
   Color.—Between Red-Orange Groups 28A and 28B.
   Thickness.—2 mm.
   Adherence to flesh.—Adheres tightly until mature.
   Ease of peeling.—Very easy when mature.
   Oil glands.—Even; not protruding.
   Scent.—Mandarin-like.
Flesh:
   Axis.—Straight.
   Puffing.—Occurs when overly mature; “puffing” is a term of art that refers to a condition where fruit segments become loosened from the interior skin surface and the fruit in its entirety becomes soft in feel and appearance.
   Number of segments.—9-10.
   Separability of segments.—Easily.
   Pulp.—Color: Orange Group 28C. Texture: Tender.
   Vesicles.—Shape: Tapered. Size: 2-4 mm in length.
Juice:
   Relative amount in fruit.—46-50%.
   Color.—Orange Group 28C.
   Aroma.—Tangerine.
   Flavor.—Tangerine-like.
   Total soluble solids.—10-13° Brix.
   Acid.—0.8-1.1%.

### REPRODUCTIVE ORGANS

Color:
   Filament.—Greyed-Orange Group 168D.
   Pollen.—Greyed-Orange Group 167A.
   Style.—Greyed-Orange Group 168D.
   Ovaries.—Greyed-Yellow Group 161B.
Seeds: Seedless under non-pollinating conditions. When present, there is an average number of 0-3 seeds per fruit colored Yellow-Green Group 145D.

### FRUIT

Persistence of fruit on tree: Hangs well.
Color:
   Aleido.—Greyed-Orange Group 170A.
   Flesh.—Greyed-Orange Group 170C.
Use: Fresh eating.
Keeping quality: 40-60 days after picking at 1-8° C.
Shipping quality: Good, if adhere to protocols.
Average amount of fruit produced per season per tree: 35 to 50 t/ha (crop yield) for a 60-90 Kg tree.
Persistence to diseases/pests: No unusual susceptibility to diseases or pests has been noted to date.

1. A new and distinct variety of Citrus reticulata plant substantially as shown and described.
